(19) United States
(12) Patent Application Publication
(10) Pub. No.: US 2026/0090776 A1
(43) Pub. Date: Apr. 2, 2026

(54) INFORMATION PROCESSING APPARATUS, LEARNING APPARATUS, INFORMATION PROCESSING METHOD, AND INFORMATION PROCESSING PROGRAM

(71) Applicant: FUJIFILM Corporation, Tokyo (JP)

(72) Inventors: Taiga GOTO, Tokyo (JP); Fuyuhiko TERAMOTO, Tokyo (JP)

(73) Assignee: FUJIFILM Corporation, Tokyo (JP)

(21) Appl. No.: 19/322,696

(22) Filed: Sep. 9, 2025

(30) Foreign Application Priority Data
Sep. 30, 2024 (JP) 2024-171853

(51) Int. Cl. A61B 6/00; A61B 6/03; A61B 6/42; A61B 6/46; G06T 5/50; G16H 30/20; G16H 30/40

(52) U.S. Cl. CPC A61B 6/482; A61B 6/032; A61B 6/4241; A61B 6/463; A61B 6/481; A61B 6/5241; G06T 5/50; G16H 30/20; G16H 30/40; G06T 2207/20224

(57) ABSTRACT

An information processing apparatus includes a processor, in which the processor acquires a plurality of pieces of imaging data corresponding to radiation having different energies, and generates a color image from the plurality of pieces of imaging data, the color image having a color assigned based on an index obtained using energy information of the radiation.

### CROSS-REFERENCE TO RELATED APPLICATION

This application claims priority under 35 USC 119 from Japanese Patent Application No. 2024-171853 filed on Sep. 30, 2024, the disclosure of which is incorporated by reference herein.

### BACKGROUND

### 1. Technical Field

The present disclosure relates to an information processing apparatus, a learning apparatus, an information processing method, and an information processing program.

### 2. Description of the Related Art

In imaging using a computed tomography (CT) apparatus, imaging methods such as multi-energy imaging and dual-energy imaging are known. In this imaging method, a plurality of pieces of imaging data corresponding to radiation having different energies are obtained. A technique is known for making various images, such as a reconstructed image generated from the imaging data, easier to diagnose. For example, JP2004-188187A describes a technique for reducing beam hardening artifacts in multi-energy CT.

### SUMMARY

However, in the related art, depending on the generated data, there are cases where a vast amount of data needs to be diagnosed, and there has been room for improvement in making the images easier to diagnose.

The present disclosure has been made in consideration of the above-described circumstances, and an object of the present disclosure is to provide an information processing apparatus, a learning apparatus, an information processing method, and an information processing program that can provide an image that is easy to diagnose.

In order to achieve the above-described object, according to a first aspect of the present disclosure, there is provided an information processing apparatus comprising: a processor, in which the processor is configured to: acquire a plurality of pieces of imaging data corresponding to radiation having different energies; and generate a color image from the plurality of pieces of imaging data, the color image having a color assigned based on an index obtained using energy information of the radiation.

According to a second aspect, in the information processing apparatus of the first aspect, the index is based on energy of the radiation, effective energy of the radiation, an effective atomic number, an electron density, or a feature value derived from the energy of the radiation, the effective energy of the radiation, the effective atomic number, or the electron density.

According to a third aspect, in the information processing apparatus of the second aspect, the processor is configured to mix pieces of imaging data corresponding to adjacent energies of the radiation into a single piece of imaging data and generate the color image.

According to a fourth aspect, in the information processing apparatus of the second aspect, the processor is configured to mix pieces of imaging data corresponding to adjacent energies of the radiation into a single piece of imaging data to reduce a difference in photon count between the pieces of imaging data, and generate the color image.

According to a fifth aspect, in the information processing apparatus of the third aspect, in a case where the color image is a color image for identifying a substance having a K absorption edge, the processor is configured to mix pieces of imaging data that are adjacent to each other in an energy direction such that the K absorption edge is not straddled.

According to a sixth aspect, in the information processing apparatus of the first aspect, the color image is a virtual monochromatic X-ray image.

According to a seventh aspect, in the information processing apparatus of the first aspect, the color image is a substance discrimination image.

According to an eighth aspect, in the information processing apparatus of the first aspect, the processor is configured to: generate a reconstructed image by reconstructing, for each color, the imaging data to which a color is assigned.

According to a ninth aspect, in the information processing apparatus of the first aspect, the processor is configured to: reconstruct the imaging data for each energy of the radiation to generate a plurality of reconstructed images; generate a difference image of the reconstructed image for each energy of the radiation; and assign a color to the difference image to generate the color image.

According to a tenth aspect, in the information processing apparatus of the first aspect, the processor is configured to: assign the color according to an interval of energy of the radiation or effective energy of the radiation.

According to an eleventh aspect, in the information processing apparatus of the first aspect, the processor is configured to: generate a composite image by combining a plurality of the color images to which different colors are assigned.

According to a twelfth aspect, in the information processing apparatus of the first aspect, the processor is configured to: display a plurality of the color images, each having a different color, under window conditions corresponding to the respective colors.

According to a thirteenth aspect, in the information processing apparatus of the first aspect, the imaging data is obtained through imaging using a contrast agent, and the processor is configured to: extract specific color information corresponding to the contrast agent; and output a time-concentration curve of the contrast agent.

According to a fourteenth aspect, in the information processing apparatus of the first aspect, the imaging data is obtained through imaging using a contrast agent, and the processor is configured to: extract specific color information corresponding to the contrast agent; estimate a time-concentration curve of the contrast agent based on the extracted color information; and specify a start timing of main imaging.

In order to achieve the above-described object, according to a fifteenth aspect of the present disclosure, there is provided a learning apparatus configured to generate an image processing model by performing machine learning on a machine learning model using training data including a set of a color image generated by the information processing apparatus according to the present disclosure and a ground truth processed image, the image processing model being configured to receive the color image as an input and output a processed image.

In order to achieve the above-described object, according to a sixteenth aspect of the present disclosure, there is provided an information processing method comprising: causing a processor to: acquire a plurality of pieces of imaging data corresponding to radiation having different energies; and generate a color image from the plurality of pieces of imaging data, the color image having a color assigned based on an index obtained using energy information of the radiation.

In order to achieve the above-described object, according to a seventeenth aspect of the present disclosure, there is provided an information processing program for causing a processor to execute a process comprising: acquiring a plurality of pieces of imaging data corresponding to radiation having different energies; and generating a color image from the plurality of pieces of imaging data, the color image having a color assigned based on an index obtained using energy information of the radiation.

According to the present disclosure, it is possible to provide an image that is easy to diagnose.

### BRIEF DESCRIPTION OF THE DRAWINGS

FIG. 1 is a configuration diagram showing an example of a configuration of a CT apparatus of an embodiment.

FIG. 2 is a configuration diagram showing an example of a configuration of a console of the embodiment.

FIG. 3 is a functional block diagram showing an example of a function of the console of the embodiment.

FIG. 4 is a flowchart showing an example of a flow of a color image generation process of the embodiment.

FIG. 5 is a diagram illustrating Example 1.

FIG. 6 is a diagram illustrating Example 1.

FIG. 7 is a diagram illustrating Example 1.

FIG. 8 is a diagram illustrating Example 1.

FIG. 9 is a diagram illustrating Example 1.

FIG. 10 is a diagram illustrating Example 2.

FIG. 11 is a diagram illustrating Example 4.

FIG. 12A is a diagram illustrating Example 5.

FIG. 12B is a diagram illustrating Example 5.

### DETAILED DESCRIPTION

Embodiments of the present invention will be described in detail below with reference to the drawings. It should be noted that the present embodiment is not intended to limit the present invention.

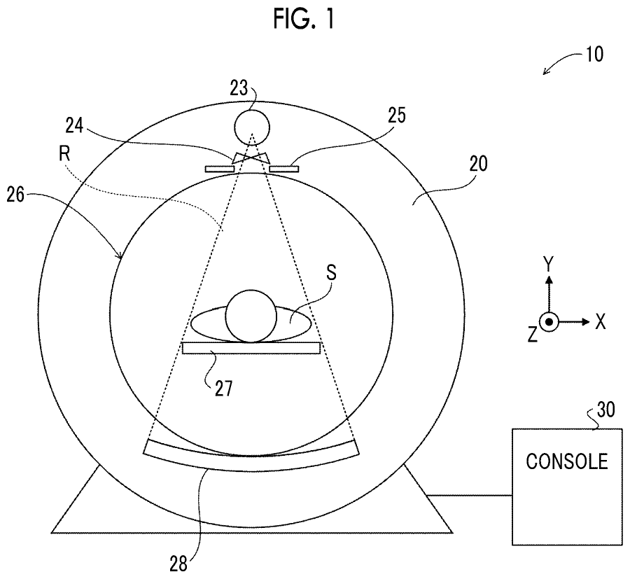

First, an example of a configuration of a radiation computed tomography (CT) imaging apparatus of the present embodiment will be described. In FIG. 1, a configuration diagram showing an example of a configuration of a CT apparatus 10 of the present embodiment is shown.

As shown in FIG. 1, the CT apparatus of the present embodiment comprises a gantry 20, a patient table 27, and a console 30. It should be noted that, in the following description, a horizontal direction in FIG. 1 is referred to as an X-axis, a vertical direction is referred to as a Y-axis, and a direction orthogonal to an XY plane is referred to as a Z-axis.

The gantry 20 has an opening portion 26, and a subject S to be imaged is disposed in the opening portion 26 in a state of being placed on the patient table 27. The gantry 20 and the patient table 27 are configured to move relative to each other in a Z-axis direction.

Inside the gantry 20, a radiation generation device 23 including a radiation tube (not shown), a bowtie filter 24, and a collimator 25, and a detector 28 are disposed to face each other with the subject S interposed therebetween. Radiation R emitted from the radiation generation device 23 is shaped by the bowtie filter 24 and the collimator 25 into a beam shape suitable for a size of the subject S and is emitted to the subject S. The detector 28 detects radiation, which has been transmitted through the subject S, and generates projection data corresponding to the dose of the detected radiation.

The radiation generation device 23 and the detector 28 are rotated around the subject S by a rotation drive unit (not shown) of the gantry 20. The radiation irradiation from the radiation generation device 23 and the radiation detection by the detector 28 are repeatedly performed while both the radiation generation device 23 and the detector 28 are rotated, thereby acquiring projection data at various projection angles. A plurality of pieces of projection data acquired by the detector 28 are reconstructed by an image reconstruction unit (not shown) of the console 30 and are output as an image.

The CT apparatus 10 of the present embodiment is a multi-energy CT. In the multi-energy CT, it is possible to acquire a plurality of pieces of projection data corresponding to radiation having different energies. As such a CT apparatus 10, for example, the detector 28 outputs projection data corresponding to photon energy. A photon counting CT comprising a photon counting detector may be used. In addition, any imaging method may be used, such as a multi-source/multi-detector method using a plurality of radiation generation devices 23 (radiation sources) and a plurality of detectors 28, a multi-layer detector method using a multi-layer detector 28 that detects radiation having different energies for each layer, a multi-scan method, an X-ray filter method, and a high-speed tube voltage switching method of varying the energy of the radiation to be emitted by switching the tube voltage according to the projection angle.

The console 30 of the present embodiment performs control related to acquisition of projection data, generation of a color image, generation of various medical images, and the like. The console 30 of the present embodiment is an example of an information processing apparatus of the present disclosure. As an example, the console 30 of the present embodiment is a server computer.

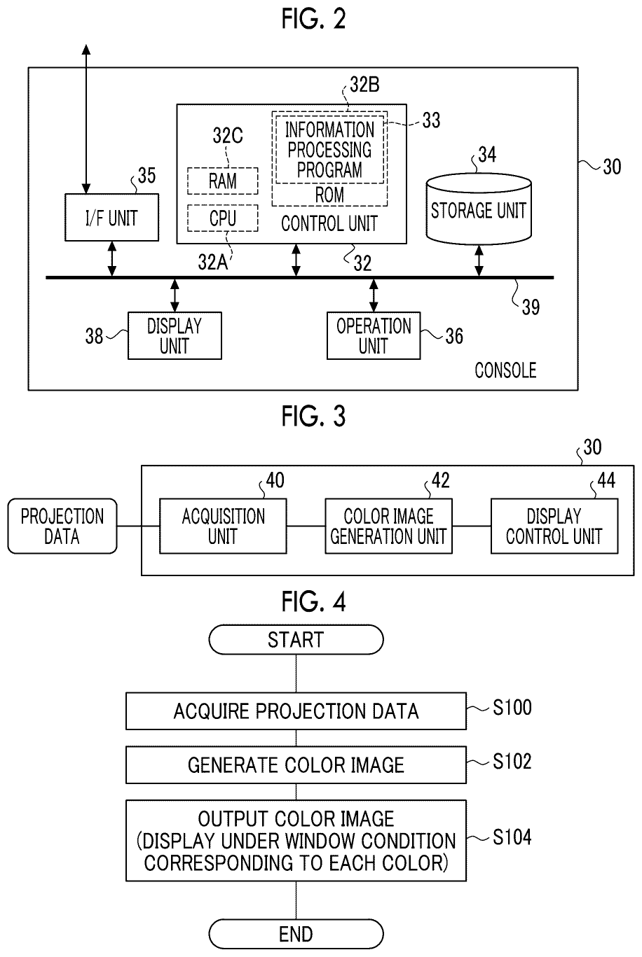

The console 30 comprises a control unit 32, a storage unit 34, an interface (I/F) unit 35, an operation unit 36, and a display unit 38, as shown in FIG. 2. The control unit 32, the storage unit 34, the I/F unit 35, the operation unit 36, and the display unit 38 are connected to each other via a bus 39, such as a system bus or a control bus, so as to be capable of exchanging various types of information.

The control unit 32 of the present embodiment controls the overall operation of the console 30. The control unit 32 comprises a central processing unit (CPU) 32A, a read-only memory (ROM) 32B, and a random access memory (RAM) 32C. The ROM 32B stores, in advance, various programs including an information processing program 33, which will be described below, to be executed by the CPU 32A, and the like. The RAM 32C temporarily stores various types of data.

The storage unit 34 stores the projection data output from the detector 28, various other types of information, and the like. As specific examples, the storage unit 34 is implemented by a storage medium such as a hard disk drive (HDD), a solid state drive (SSD), and a flash memory.

The I/F unit 35 performs communication of various types of information with the rotation drive unit (not shown) of the gantry 20, the radiation generation device 23, and the detector 28 through wired communication or wireless communication. The console 30 of the present embodiment receives the projection data from the detector 28 via the I/F unit 35. The received projection data is stored in the storage unit 34 in association with the projection angle and the energy of the radiation.

The operation unit 36 is used by the user to input scan conditions for acquiring projection data, instructions related to generation, display, and the like of images, or various types of information, and the like. The operation unit 36 is not particularly limited, and examples thereof include various switches, buttons, a touch panel, a touch pen, a keyboard, and a mouse. The display unit 38 displays various types of information, a medical image, and the like. It should be noted that the operation unit 36 and the display unit 38 may be integrated into a touch panel display. Additionally, for example, the operation unit 36 may receive a voice input from the user.

In FIG. 3, a functional block diagram showing an example of a function of the console 30 is shown. The console 30 comprises an acquisition unit 40, a color image generation unit 42, and a display control unit 44. As an example, in the console 30 of the present embodiment, the CPU 32A of the control unit 32 executes the information processing program 33, whereby the CPU 32A functions as the acquisition unit 40, the color image generation unit 42, and the display control unit 44.

The acquisition unit 40 has a function of acquiring a plurality of pieces of projection data corresponding to radiation having different energies. It should be noted that the acquisition unit 40 may acquire the projection data from the detector 28 or may acquire the projection data from the storage unit 34 in a case where the projection data is stored in the storage unit 34 in advance. The acquisition unit 40 outputs the acquired projection data to the color image generation unit 42. The projection data of the present embodiment is an example of imaging data of the present disclosure. It should be noted that the imaging data of the present disclosure is not limited to the projection data and may be, for example, image data, scanographic data, or the like.

The color image generation unit 42 has a function of generating, from a plurality of pieces of projection data, a color image to which a color is assigned based on an index obtained using energy information of radiation. The index is based on energy of the radiation, effective energy of the radiation, an effective atomic number, an electron density, or a feature value derived from the energy of the radiation, the effective energy of the radiation, the effective atomic number, or the electron density. Which of these is used as the index may be determined according to the user's designation, or according to the imaging method, the purpose of the imaging or diagnosis, or the like.

In addition, the image to be a color image, that is, the image to which a color is assigned, may be an image obtained by processing the projection data, such as a reconstructed image, or may be an image obtained by further processing the image obtained by processing the projection data. Further, the color image generation unit may generate the color image by assigning a color to the image generated from the projection data or may generate the color image by using the projection data to which a color is assigned.

The type of color to be assigned may be predetermined, may be determined according to the index, or may be determined according to the user's designation. Additionally, the type of color may also be determined according to an output destination of the color image. For example, in a case where the image is displayed on the display unit 38, the color may be based on RGB, and in a case where the image is output to a printer or the like to form a printed material, the color may be based on CMYK.

The color image generation unit 42 outputs the generated color image to the display control unit 44.

The display control unit 44 has a function of performing control to display the color image generated by the color image generation unit 42 on the display unit 38. In a case where the color image is not displayed on the display unit 38, for example, in a case where the color image is output to an external device, the display control unit 44 outputs image data corresponding to the color image to the output destination.

Next, an operation of the console 30 of the present embodiment will be described.

In a case where the console 30 of the present embodiment receives, as an example, an instruction to output the color image, which is input by the user through the operation unit 36, the CPU 32A of the control unit 32 executes the information processing program 33 stored in the ROM 32B to execute a color image generation process shown in FIG. 4 as an example. FIG. 4 is a flowchart showing an example of a flow of the color image generation process in the console 30 of the present embodiment.

First, in step S100 of FIG. 4, the acquisition unit 40 acquires a plurality of pieces of projection data corresponding to the radiation having different energies as mentioned above.

In the next step S102, as mentioned above, the color image generation unit 42 generates the color image to which a color is assigned based on the index obtained using the energy information of the radiation, from the plurality of pieces of projection data acquired in step S100 described above.

In the next step S104, as mentioned above, the display control unit 44 outputs the color image generated in step S102 described above. As an example, in the present embodiment, control is performed to output the color image to the display unit 38 and display the color image on the display unit 38.

In the color images of the respective colors, the captured content differs depending on the color, and the contrast also differs. Therefore, in the present embodiment, in a case where a color image is to be displayed, the color image is displayed under window conditions corresponding to the respective colors, that is, window conditions corresponding to the contrast. As an example, the smaller the contrast is, the narrower the window is. Specifically, the display control unit 44 sets the window conditions (a window width (WW) and a window level (WL)) independently for each color. In the present embodiment, the window width for each color is referred to as a reference window width, the window level for each color is referred to as a reference window level, and these window conditions are referred to as reference window conditions. The window condition may be converted by a linear function according to each color. For example, each color is independently set by multiplying the reference window width by a coefficient and adding an offset to the reference window level. In addition, as the method for setting the window conditions, preset values may be stored in advance according to combinations of base materials, types of examinations or diagnoses, or diagnostic purposes and may be selected or changed by the user. Further, the energy of the radiation corresponding to the color image and the window width may also be linked such that the higher the energy of the radiation corresponding to the color image is, the smaller the window width is.

In a case where the processing of step S104 ends, the color image generation process shown in FIG. 4 ends.

Furthermore, specific examples of the generation of the color image and the like by the console 30 will be described.

### Example 1

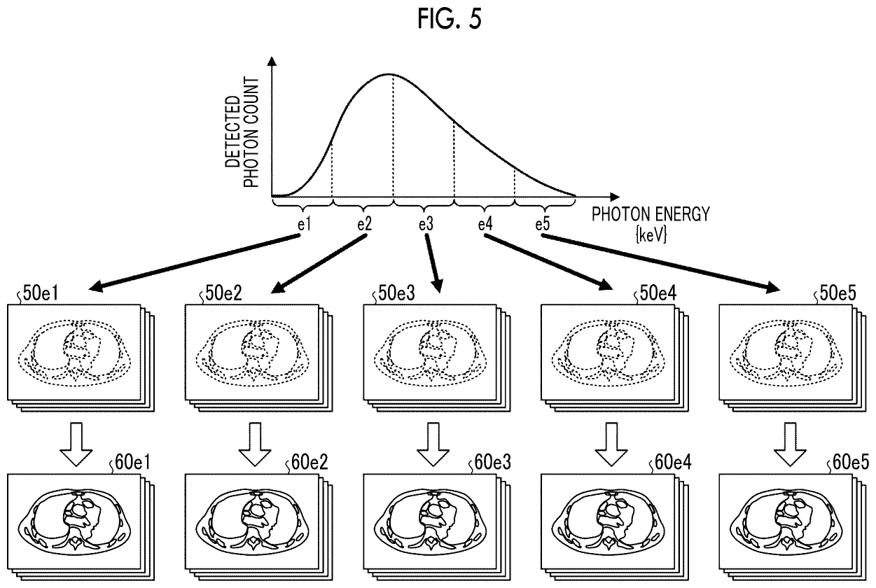

In the present example, a case will be described in which the CT apparatus 10 is a photon counting CT. As shown in FIG. 5, with the CT apparatus 10, a plurality of pieces of projection data 50e1 to 50e5 are obtained respectively for five bands (energy bands) of the energy (photon energy) of the radiation. The color image generation unit 42 of the console 30 obtains a reconstructed image 60e1 by reconstructing the plurality of pieces of projection data 50e1 corresponding to energy e1 of the radiation and obtains a reconstructed image 60e2 by reconstructing the plurality of pieces of projection data 50e2 corresponding to energy e2 of the radiation. Additionally, the color image generation unit 42 obtains a reconstructed image 60e3 by reconstructing the plurality of pieces of projection data 50e3 corresponding to energy e3 of the radiation, obtains a reconstructed image 60e4 by reconstructing the plurality of pieces of projection data 50e4 corresponding to energy e4 of the radiation, and obtains a reconstructed image 60e5 by reconstructing the plurality of pieces of projection data 50e5 corresponding to energy e5 of the radiation. The color image generation unit 42 may generate the color image by assigning different colors to the reconstructed images 60e1 to 60e5.

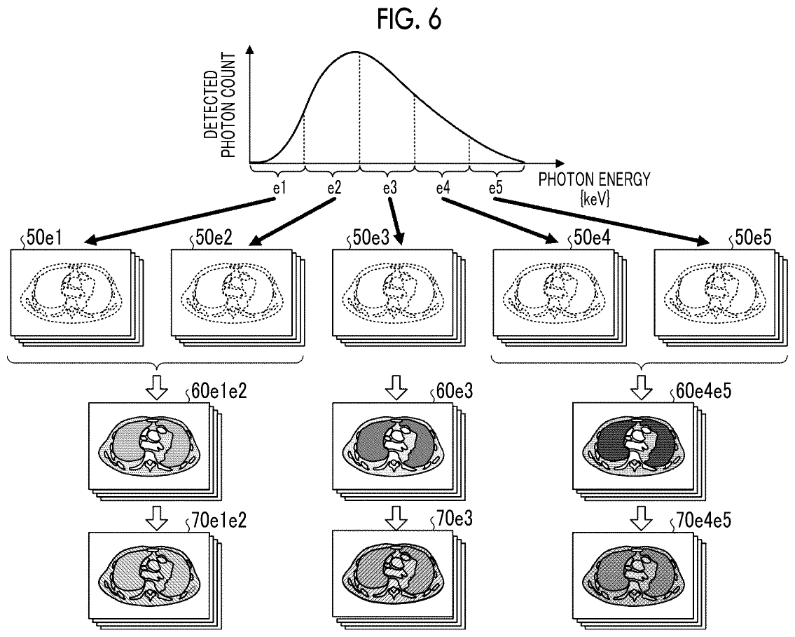

In addition, there may be cases where the number of colors to be assigned is fixed or there is a bias in the photon count for each energy band. In such a case, the color image generation unit 42 mixes pieces of projection data 50 corresponding to adjacent energies of the radiation and generates the color image as a single piece of projection data 50. As an example, as shown in FIG. 6, the color image generation unit 42 of the present example reconstructs a projection data group obtained by mixing the plurality of pieces of projection data 50e1 corresponding to the energy e1 of the radiation and the plurality of pieces of projection data 50e2 corresponding to the energy e2 of the radiation to generate a reconstructed image 60e1e2 and assigns a color, for example, red (R), to the reconstructed image 60e1e2 to generate a color image 70e1e2. Further, the color image generation unit 42 reconstructs the plurality of pieces of projection data 50e3 corresponding to the energy e3 of the radiation to generate the reconstructed image 60e3 and assigns a color, for example, green (G), to the reconstructed image 60e3 to generate a color image 70e3. Additionally, the color image generation unit 42 reconstructs a projection data group obtained by mixing the plurality of pieces of projection data 50e4 corresponding to the energy e4 of the radiation and the plurality of pieces of projection data 50e5 corresponding to the energy e5 of the radiation to generate a reconstructed image 60e4e5 and assigns a color, for example, blue (B), to the reconstructed image 60e4e5 to generate a color image 70e4e5. Each of the color image 70e1e2, the color image 70e3, and the color image 70e4e5 is an image having a color density corresponding to a pixel value.

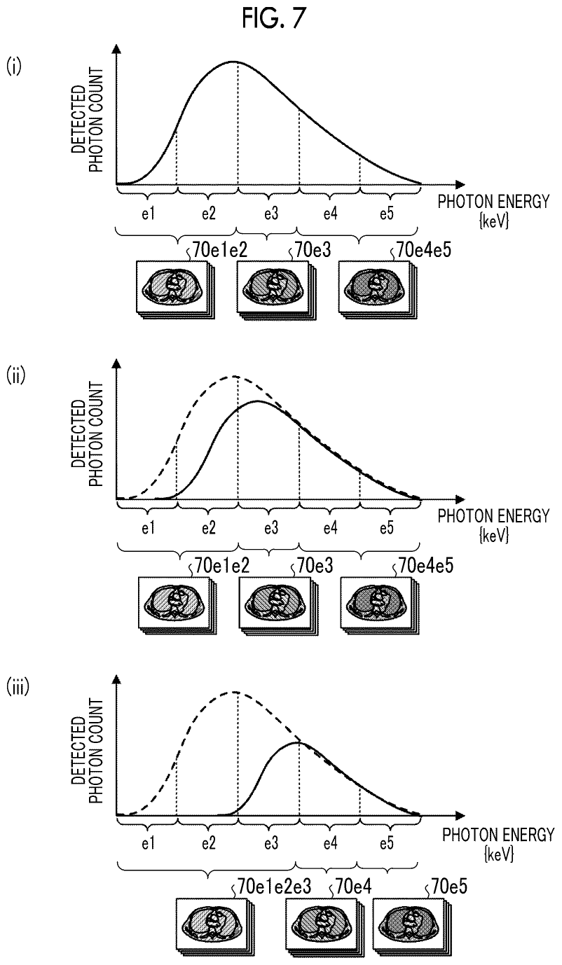

It should be noted that which pieces of projection data 50 corresponding to which energy are to be mixed may be predetermined or may be varied dynamically. For example, as shown in FIG. 7, the photon count detected by the detector 28 changes according to the thickness of the subject S. In the example shown in FIG. 7, in case (i), where the thickness of the subject S is average, the color image generation unit 42 finally generates the color image 70e1e2 from the projection data group obtained by mixing the plurality of pieces of projection data 50e1 and 50e2 respectively corresponding to the energies e1 and e2 of the radiation, as mentioned above. In addition, the color image generation unit 42 finally generates the color image 70e3 from the plurality of pieces of projection data 50e3 corresponding to the energy e3 of the radiation. Further, the color image generation unit 42 finally generates the color image 70e4e5 from the projection data group obtained by mixing the plurality of pieces of projection data 50e4 and 50e5 respectively corresponding to the energies e4 and e5 of the radiation. In case (ii), where the thickness of the subject S is slightly less than in case (i), the photon count detected by the detector 28 decreases according to the thickness. In case (ii), in consideration of the photon counts corresponding to the energies e1 to e5, the color image generation unit 42 generates the color images 70e1e2, 70e3, and 70e4e5, in the same manner as in case (i) described above. In case (iii), where the thickness of the subject S is less than in case (ii), the photon count detected by the detector 28 further decreases according to the thickness. In case (iii), in consideration of the photon counts corresponding to the energies e1 to e5, the color image generation unit 42 finally generates the color image 70e1e2e3 from the projection data group obtained by mixing the plurality of pieces of projection data 50e1 to 50e3 respectively corresponding to the energies e1 to e3 of the radiation. Additionally, the color image generation unit 42 finally generates a color image 70e4 from the plurality of pieces of projection data 50e4 corresponding to the energy e4 of the radiation. Further, the color image generation unit 42 finally generates a color image 70e5 from the plurality of pieces of projection data 50e5 corresponding to the energy e5 of the radiation.

In this way, for the plurality of pieces of projection data 50 used to generate the reconstructed image, by mixing the pieces of projection data 50 corresponding to adjacent energies such that the difference in the photon count between the energies (energy bands) is small, it is possible to improve the S/N ratio of a reconstructed image 60 and a color image 70.

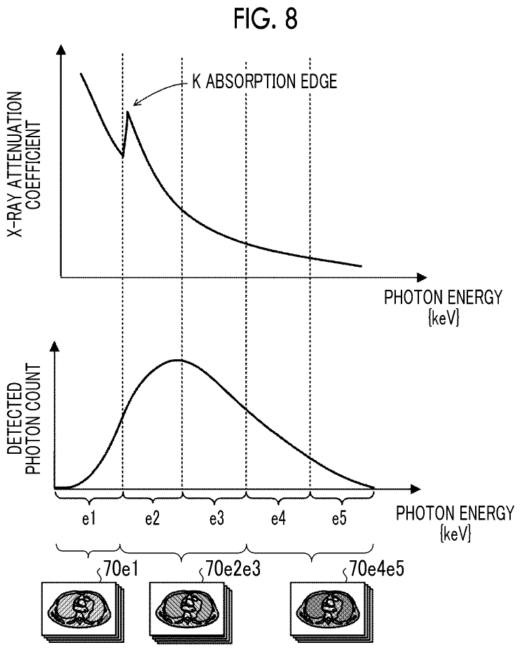

In addition, in a case where the color image 70 is a color image for identifying a substance having a K absorption edge (K-edge), that is, in a case where the substance to be identified by a person who interprets medical images has a K absorption edge, the color image generation unit 42 may mix pieces of projection data 50 that are adjacent to each other in an energy direction such that the K absorption edge is not straddled. In the example shown in FIG. 8, mixing the plurality of pieces of projection data 50e1 and 50e2 respectively corresponding to the energies e1 and e2 of the radiation results in straddling the K absorption edge. Therefore, in the case shown in FIG. 8, the color image generation unit 42 finally generates a color image 70e1 from the plurality of pieces of projection data 50e1 corresponding to the energy e1 of the radiation. Further, a color image 70e2e3 is finally generated from the projection data group obtained by mixing the plurality of pieces of projection data 50e2 and 50e3 respectively corresponding to the energies e2 and e3 of the radiation. Furthermore, the color image generation unit 42 finally generates the color image 70e4e5 from the projection data group obtained by mixing the plurality of pieces of projection data 50e4 and 50e5 respectively corresponding to the energies e4 and e5 of the radiation.

In this way, by mixing the pieces of projection data 50 such that the K absorption edge is not straddled, it is possible to suppress the difficulty in recognizing the effects caused by the K absorption edge.

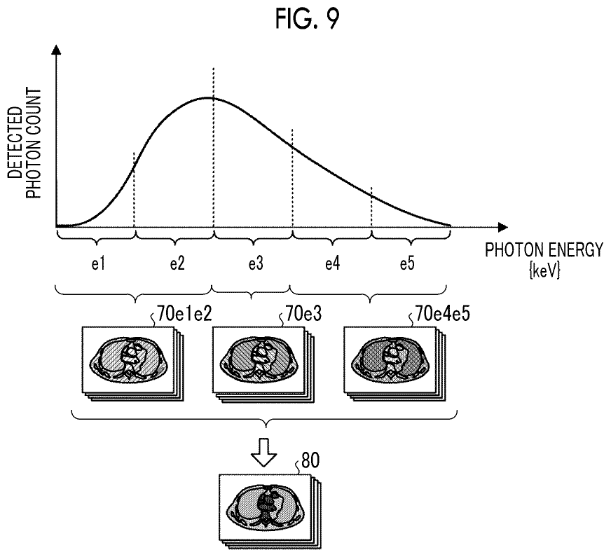

In the CT apparatus 10 of the present example, the color image generation unit 42 may generate a composite image by combining a plurality of color images 70 to which different colors are assigned. For example, as shown in FIG. 9, in a case where the color image 70e1e2 to which R is assigned, the color image 70e3 to which G is assigned, and the color image 70e4e5 to which B is assigned are generated, the color image generation unit 42 generates a composite image 80, which is an RGB color image, by combining the color images 70e1e2, 70e3, and 70e4e5. With the composite image 80 which is the color image obtained by combining the color images 70, identification of substances becomes easier based on differences in color.

In this way, with the console 30 of the present example, it is possible to provide an image that is easy to diagnose.

### Example 2

In Example 1, a case has been described in which the CT apparatus 10 is a photon counting CT. In the present example, a case will be described in which the CT apparatus 10 performs multi-energy imaging by varying the energy of the radiation reaching the detector 28.

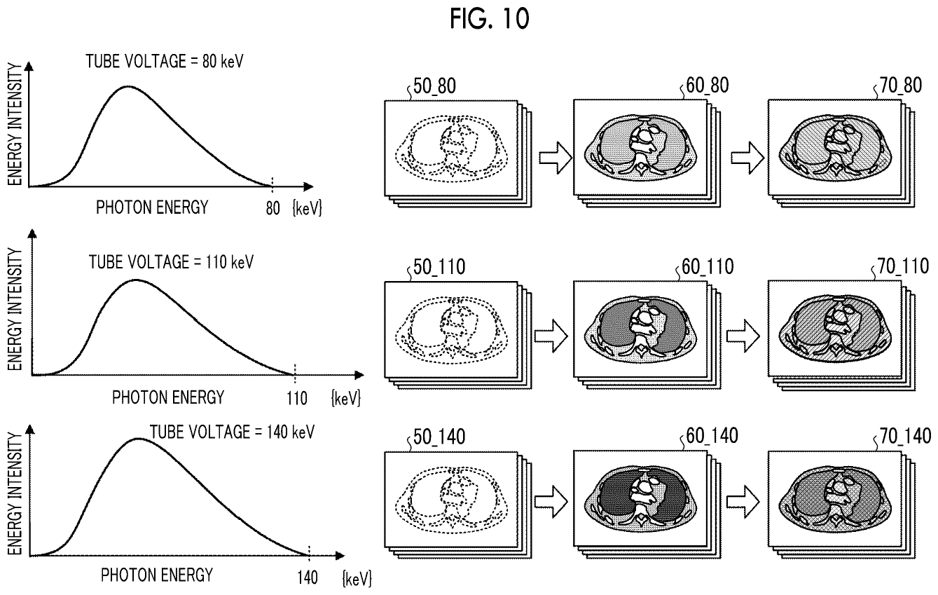

As shown in FIG. 10, with the CT apparatus 10, a plurality of pieces of projection data 50_80 are obtained through imaging performed with a tube voltage of 80 kV. The color image generation unit 42 of the console 30 obtains a reconstructed image 60_80 by reconstructing the plurality of pieces of projection data 50_80 and assigns a color (for example, R) to the reconstructed image 60_80 to generate a color image 70_80. Additionally, a plurality of pieces of projection data 50_110 are obtained through imaging performed with a tube voltage of 110 kV. The color image generation unit 42 of the console 30 obtains a reconstructed image 60_110 by reconstructing the plurality of pieces of projection data 50_110 and assigns a color (for example, G) to the reconstructed image 60_110 to generate a color image 70_110. Further, a plurality of pieces of projection data 50_140 are obtained through imaging performed with a tube voltage of 140 kV. The color image generation unit 42 of the console 30 obtains a reconstructed image 60_140 by reconstructing the plurality of pieces of projection data 50_140 and assigns a color (for example, B) to the reconstructed image 60_140 to generate a color image 70_140.

In this way, in the console 30 of the present example, the color image 70 can also be generated in the same manner as in Example 1. Accordingly, in the console 30 of the present example, it is also possible to provide an image that is easy to diagnose.

### Example 3

The color image 70 may be a virtual monochromatic X-ray image, and the color to be assigned may vary depending on the energy of the radiation. For example, the color image 70, which is the virtual monochromatic X-ray image, may be generated from the projection data 50 corresponding to the energy of 40 keV, the color image 70, which is the virtual monochromatic X-ray image, may be generated from the projection data 50 corresponding to the energy of 70 keV, and the color image 70, which is the virtual monochromatic X-ray image, may be generated from the projection data 50 corresponding to the energy of 100 keV. By combining these three types of color images 70 to which different colors are assigned, the visibility of the composite image 80 can be improved. In this case, the energy used for assigning the color may be designated by the user.

In addition, the color image 70 may be a substance discrimination image, and the color to be assigned may vary depending on a reference substance. For example, in a case where the reference substance is water, a bone, and iodine (contrast agent), a color image 70, which is a substance discrimination image in which the reference substance is water, may be generated, a color image 70, which is a substance discrimination image in which the reference substance is a bone, may be generated, and a color image 70, which is a substance discrimination image in which the reference substance is iodine, may be generated. By combining these three types of color images 70 to which different colors are assigned, the visibility of the composite image 80 can be improved. In this case, the reference substance for assigning the color may be designated by the user.

In this way, in the console 30 of the present example, it is also possible to provide an image that is easy to diagnose.

### Example 4

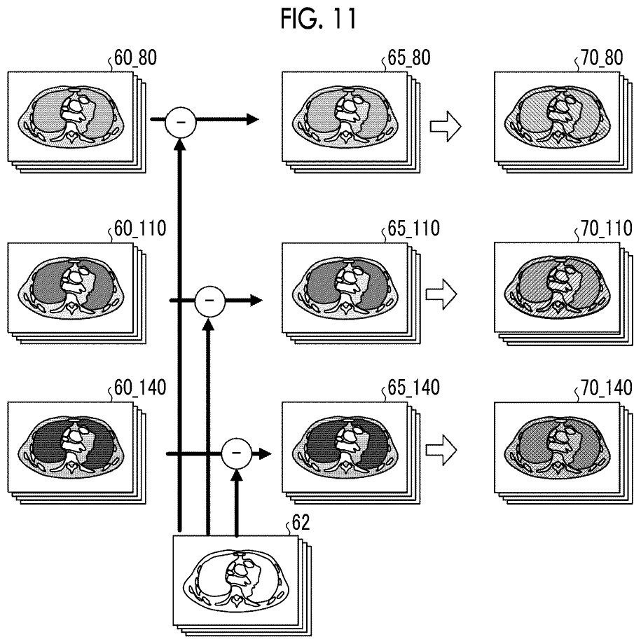

The console 30 may generate a difference image of the reconstructed image 60 for each energy of the radiation and may generate the color image 70 by assigning a color to the difference image. In the example shown in FIG. 11, the color image generation unit 42 generates difference images 65_80, 65_110, and 65_140 for the respective reconstructed images 60_80, 60_110, and 60_140 described in Example 2. The difference image 65_80 is a difference image between a reference image 62 and the reconstructed image 60_80. The difference image 65_110 is a difference image between the reference image 62 and the reconstructed image 60_110. The difference image 65_140 is a difference image between the reference image 62 and the reconstructed image 60_140. The reference image 62 may be an image obtained by performing predetermined processing on the reconstructed images 60_80, 60_110, and 60_140. Examples of the image in this case include an image obtained by averaging the pixel values of each reconstructed image 60. Additionally, the reference image 62 may be any of the reconstructed image 60_80, 60_110, or 60_140, and the reference image 62 may be varied depending on which of the reconstructed images 60_80, 60_110, and 60_140 is used as a target for difference calculation.

The color image generation unit 42 generates color images 70_80, 70_110, and 70_140 by assigning different colors to the difference images 65_80, 65_110, and 65_140, respectively. By combining the color images 70_80, 70_110, and 70_140, the visibility of the composite image 80 can be improved.

In this way, in the console 30 of the present example, it is also possible to provide an image that is easy to diagnose.

### Example 5

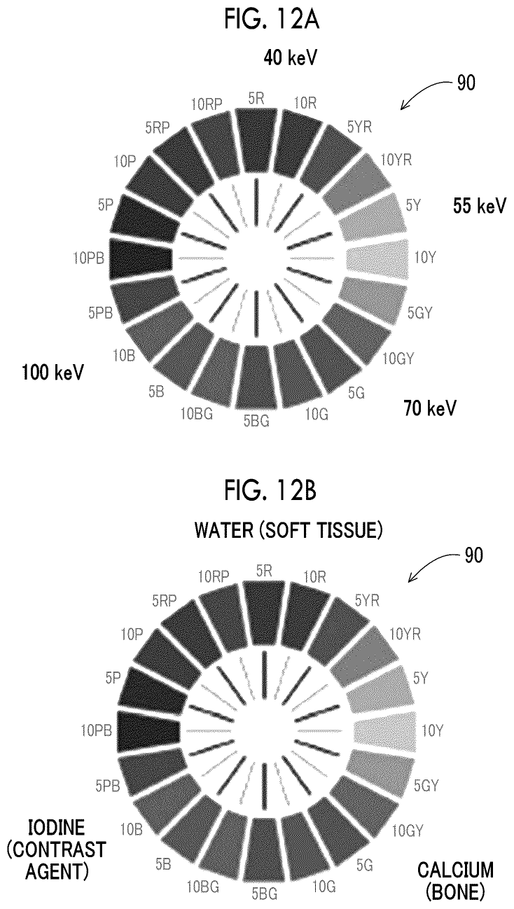

The color image generation unit 42 of the console 30 may generate the color image 70 by assigning a color corresponding to the interval of the energy of the radiation or the effective energy of the radiation. FIGS. 12A and 12B show a method for the color image generation unit 42 to specify a color corresponding to the interval of the energy of the radiation. In the example shown in FIG. 12A, an example of associating the energy of the radiation with a hue circle 90 is shown. The color image generation unit 42 associates the reference energy with the reference color in the hue circle 90. The color image generation unit 42 assigns a color to the desired energy of the radiation by associating the interval of the energy with the hue circle 90.

As shown in FIG. 12B, the color image generation unit 42 may assign colors to reference substances (water, iodine, and calcium in FIG. 12B) with the hue circle 90 or the like as a reference. By assigning the colors in this way, the colors corresponding to the characteristics of the image can be assigned. The color image generation unit 42 may assign colors in association with the hue circle 90 with the effective atomic number as a reference.

### Example 6

In the present example, a case will be described in which the CT apparatus 10 obtains the projection data 50 through imaging using a contrast agent.

The color image generation unit 42 of the console 30 generates the color image 70 by assigning a color for each energy (Bin) of the radiation for the reconstructed image 60. A case will be described in which red is assigned to a first Bin having the lowest photon energy, green is assigned to a second Bin having an intermediate energy, and blue is assigned to a third Bin having the highest photon energy. In a case where the contrast agent is iodine, the lower the energy is, the higher the CT value is. Accordingly, the image appears reddish. This is in the same manner as calcium. In addition, in a case where the contrast agent is gadolinium, the CT value in the second Bin increases. Accordingly, the image appears greenish. Therefore, it is easier to distinguish between gadolinium and iodine. Further, in the case of acrylic, the higher the energy is, the higher the CT value is. Accordingly, the image appears bluish. This is in the same manner as fat. Additionally, in the case of water, a constant CT value is exhibited regardless of the energy. Accordingly, the image appears grayish (black and white).

In this way, since the colors appear differently depending on the contrast agent or tissue, the console 30 can obtain various types of information by extracting specific color information. For example, the console 30 may extract specific color information corresponding to the contrast agent and generate a time-concentration curve of the contrast agent.

In addition, for example, the console 30 may extract the specific color information corresponding to the contrast agent, estimate the time-concentration curve of the contrast agent, and specify a start timing of main imaging of contrast imaging based on an estimation result. Specifically, during bolus tracking, the specific color information corresponding to the contrast agent may be extracted, and a timing at which the concentration of the contrast agent reaches a preferable state may be specified as the start timing of the main imaging.

It should be noted that it is more effective in a case where the aspect of the present example is applied to bolus tracking using two types of contrast agents: gadolinium and iodine.

### Example 7

The console 30 may generate an image processing model that receives the color image 70 as an input and that outputs a processed image, by performing machine learning on a machine learning model using training data including a set of the generated color image 70 and a ground truth processed image. Examples of the processed image in this case include a denoised image, a segmented image, and an artifact-corrected image. In order to achieve highly accurate learning, it is preferable that the training data corresponding to the input color data is also color data. Additionally, it is preferable that the information content (for example, the energy (keV), the effective atomic number, or the reference substance) corresponding to the hue of the input data is made to match the information content corresponding to the hue of the training data. Further, it is also preferable to match the number of colors with that used during training by correcting at least one of internal parameters of the network (for example, a parameter of the activation function) according to the number of colors in the color data, or by setting a value of zero to an unused color. In this way, by correcting the parameter of the activation function according to the described number of colors, it is possible to prevent erroneous results by reducing the output of the activation function corresponding to the color that has not been input. The network may be switched according to the number of colors of the color image 70. The console 30 in the present example is an example of a learning apparatus of the present disclosure.

According to the present example, it is possible to generate an image processing model that can output a processed image with high accuracy.

As described above, with the console 30 of each of the above-described embodiments, it is possible to provide an image that is easy to diagnose.

In addition, in the present embodiment, each process is executed by any computer. Further, any computer may execute these processes by means of a processor as hardware, a program as software, or a combination thereof. In that case, the processor is configured to execute various types of processes in the present embodiment in cooperation with the program, and can function as each unit or each means in the present embodiment. Furthermore, the execution order of the process by the processor is not limited to the order described above and may be changed as appropriate. Any computer may be a general-purpose computer, a computer for a specific application, a workstation, or another system capable of executing each process.

The processor may be configured using one or more pieces of hardware, and the type of hardware is not limited. For example, the processor can be configured using hardware such as a central processing unit (CPU), a micro processing unit (MPU), a programmable logic device such as a field programmable gate array (FPGA), a dedicated circuit for executing specific processing such as an application specific integrated circuit (ASIC), a graphic processing unit (GPU) or a neural processing unit (NPU). Additionally, the type of hardware may be a combination of different types of hardware. In a case where a plurality of pieces of hardware are configured to execute one or more processes of a certain processor, the plurality of pieces of hardware may be present in devices physically separated from each other or may be present in the same device. Further, in any of the embodiments, the order of each process by the processor is not limited to the order described above and may be changed as appropriate. The hardware is configured using an electrical circuit (circuitry) formed by combining circuit elements such as semiconductor elements, or the like.

Furthermore, the program may be software such as firmware or a microcode. Moreover, the program may be, for example, a program module group, and each function thereof may be implemented by a processor configured to execute the corresponding function. The program may be a program code or a plurality of code segments stored in one or a plurality of non-transitory computer-readable media (for example, storage media, other storages, or the like). The program may be stored in a distributed manner across a plurality of non-transitory computer-readable media that are present in devices physically separated from each other. The program code or code segments may represent any combination of procedures, functions, subprograms, routines, subroutines, modules, software packages, classes, or commands, data structures, or program statements. The program code or code segments may be connected to other code segments or hardware circuits by transmitting and receiving information, data, arguments, parameters, or contents of a memory.

Additionally, in the above-described embodiments, an aspect has been described in which the information processing program 33 is stored (installed) in advance in the ROM 32B, but the present disclosure is not limited to this aspect. The information processing program 33 may be provided in a form recorded on a recording medium such as a compact disc read-only memory (CD-ROM), a digital versatile disc read-only memory (DVD-ROM), and a universal serial bus (USB) memory. Alternatively, the information processing program 33 may be provided in a form that can be downloaded from an external device via a network.

In addition, the technology of the present disclosure extends to all program products. The program product includes all forms of products for providing a program. For example, the program product includes a program provided through a network such as the Internet, a non-transitory computer-readable recording medium such as a CD-ROM, a DVD, and a USB memory in which the program is stored, and the like.

Additionally, it is obvious that the configurations, operations, and the like of the CT apparatus 10, the console 30, and the like described in each of the above-described embodiments are merely examples and can be changed depending on the situation within the scope of the present invention without departing from its gist. Further, it is obvious that the above-described embodiments may be combined as appropriate.

In addition, the present invention can also be applied to a program and a program product.

The following supplementary notes are disclosed with respect to the above-described embodiments.

### Supplementary Note 1

An information processing apparatus comprising:
a processor,
in which the processor is configured to:
   acquire a plurality of pieces of imaging data corresponding to radiation having different energies; and
   generate a color image from the plurality of pieces of imaging data, the color image having a color assigned based on an index obtained using energy information of the radiation.

### Supplementary Note 2

The information processing apparatus according to Supplementary Note 1,
in which the index is based on energy of the radiation, effective energy of the radiation, an effective atomic number, an electron density, or a feature value derived from the energy of the radiation, the effective energy of the radiation, the effective atomic number, or the electron density.

### Supplementary Note 3

The information processing apparatus according to Supplementary Note 1 or 2,
in which the processor is configured to mix pieces of imaging data corresponding to adjacent energies of the radiation into a single piece of imaging data and generate the color image.

### Supplementary Note 4

The information processing apparatus according to Supplementary Note 1 or 2,
in which the processor is configured to mix pieces of imaging data corresponding to adjacent energies of the radiation into a single piece of imaging data to reduce a difference in photon count between the pieces of imaging data, and generate the color image.

### Supplementary Note 5

The information processing apparatus according to Supplementary Note 3 or 4,
in which, in a case where the color image is a color image for identifying a substance having a K absorption edge,
the processor is configured to mix pieces of imaging data that are adjacent to each other in an energy direction such that the K absorption edge is not straddled.

### Supplementary Note 6

The information processing apparatus according to any one of Supplementary Notes 1 to 5,
in which the color image is a virtual monochromatic X-ray image.

### Supplementary Note 7

The information processing apparatus according to any one of Supplementary Notes 1 to 5,
in which the color image is a substance discrimination image.

### Supplementary Note 8

The information processing apparatus according to any one of Supplementary Notes 1 to 5,
in which the processor is configured to:
   generate a reconstructed image by reconstructing, for each color, the imaging data to which a color is assigned.

### Supplementary Note 9

The information processing apparatus according to any one of Supplementary Notes 1 to 5,
in which the processor is configured to:
   reconstruct the imaging data for each energy of the radiation to generate a plurality of reconstructed images;
   generate a difference image of the reconstructed image for each energy of the radiation; and
   assign a color to the difference image to generate the color image.

### Supplementary Note 10

The information processing apparatus according to any one of Supplementary Notes 1 to 9,
in which the processor is configured to:
   assign the color according to an interval of energy of the radiation or effective energy of the radiation.

### Supplementary Note 11

The information processing apparatus according to any one of Supplementary Notes 1 to 10,
in which the processor is configured to:
   generate a composite image by combining a plurality of the color images to which different colors are assigned.

### Supplementary Note 12

The information processing apparatus according to any one of Supplementary Notes 1 to 11,
in which the processor is configured to:
   display a plurality of the color images, each having a different color, under window conditions corresponding to the respective colors.

### Supplementary Note 13

The information processing apparatus according to Supplementary Note 1,
in which the imaging data is obtained through imaging using a contrast agent, and
the processor is configured to:
   extract specific color information corresponding to the contrast agent; and
   output a time-concentration curve of the contrast agent.

### Supplementary Note 14

The information processing apparatus according to Supplementary Note 1,
in which the imaging data is obtained through imaging using a contrast agent, and
the processor is configured to:
   extract specific color information corresponding to the contrast agent;
   estimate a time-concentration curve of the contrast agent based on the extracted color information; and
   specify a start timing of main imaging.

### Supplementary Note 15

A learning apparatus configured to generate an image processing model by performing machine learning on a machine learning model using training data including a set of a color image generated by the information processing apparatus according to any one of Supplementary Notes 1 to 14 and a ground truth processed image, the image processing model being configured to receive the color image as an input and output a processed image.

### Supplementary Note 16

An information processing method comprising:
causing a processor to:
   acquire a plurality of pieces of imaging data corresponding to radiation having different energies; and
   generate a color image from the plurality of pieces of imaging data, the color image having a color assigned based on an index obtained using energy information of the radiation.

### Supplementary Note 17

An information processing program for causing a processor to execute a process comprising:
acquiring a plurality of pieces of imaging data corresponding to radiation having different energies; and
generating a color image from the plurality of pieces of imaging data, the color image having a color assigned based on an index obtained using energy information of the radiation.

What is claimed is:

1. An information processing apparatus comprising:
a processor,
wherein the processor is configured to:
acquire a plurality of pieces of imaging data corresponding to radiation having different energies; and
generate a color image from the plurality of pieces of imaging data, the color image having a color assigned based on an index obtained using energy information of the radiation.

2. The information processing apparatus according to claim 1,
wherein the index is based on energy of the radiation, effective energy of the radiation, an effective atomic number, an electron density, or a feature value derived from the energy of the radiation, the effective energy of the radiation, the effective atomic number, or the electron density.

3. The information processing apparatus according to claim 2,
wherein the processor is configured to mix pieces of imaging data corresponding to adjacent energies of the radiation into a single piece of imaging data and generate the color image.

4. The information processing apparatus according to claim 2,
wherein the processor is configured to mix pieces of imaging data corresponding to adjacent energies of the radiation into a single piece of imaging data to reduce a difference in photon count between the pieces of imaging data, and generate the color image.

5. The information processing apparatus according to claim 3,
wherein, in a case where the color image is a color image for identifying a substance having a K absorption edge,
the processor is configured to:
mix pieces of imaging data that are adjacent to each other in an energy direction such that the K absorption edge is not straddled.

6. The information processing apparatus according to claim 1,
wherein the color image is a virtual monochromatic X-ray image.

7. The information processing apparatus according to claim 1,
wherein the color image is a substance discrimination image.

8. The information processing apparatus according to claim 1,
wherein the processor is configured to:
generate a reconstructed image by reconstructing, for each color, the imaging data to which a color is assigned.

9. The information processing apparatus according to claim 1,
wherein the processor is configured to:
reconstruct the imaging data for each energy of the radiation to generate a plurality of reconstructed images;
generate a difference image of the reconstructed image for each energy of the radiation; and
assign a color to the difference image to generate the color image.

10. The information processing apparatus according to claim 1,
wherein the processor is configured to:
assign the color according to an interval of energy of the radiation or effective energy of the radiation.

11. The information processing apparatus according to claim 1,
wherein the processor is configured to:
generate a composite image by combining a plurality of the color images to which different colors are assigned.

12. The information processing apparatus according to claim 1,
wherein the processor is configured to:
display a plurality of the color images, each having a different color, under window conditions corresponding to the respective colors.

13. The information processing apparatus according to claim 1,
wherein the imaging data is obtained through imaging using a contrast agent, and
the processor is configured to:
extract specific color information corresponding to the contrast agent; and
output a time-concentration curve of the contrast agent.

14. The information processing apparatus according to claim 1,
wherein the imaging data is obtained through imaging using a contrast agent, and
the processor is configured to:
extract specific color information corresponding to the contrast agent;
estimate a time-concentration curve of the contrast agent based on the extracted color information; and
specify a start timing of main imaging.

15. A learning apparatus configured to generate an image processing model by performing machine learning on a machine learning model using training data including a set of a color image generated by the information processing apparatus according to claim 1 and a ground truth processed image, the image processing model being configured to receive the color image as an input and output a processed image.

16. An information processing method comprising:
causing a processor to:
acquire a plurality of pieces of imaging data corresponding to radiation having different energies; and
generate a color image from the plurality of pieces of imaging data, the color image having a color assigned based on an index obtained using energy information of the radiation.

17. A non-transitory computer-readable storage medium storing an information processing program for causing a processor to execute a process comprising:
acquiring a plurality of pieces of imaging data corresponding to radiation having different energies; and
generating a color image from the plurality of pieces of imaging data, the color image having a color assigned based on an index obtained using energy information of the radiation.
